(19) United States
(12) Patent Application Publication
(10) Pub. No.: US 2017/0095456 A1
(43) Pub. Date: Apr. 6, 2017

(54) Therapeutic and Method of Use

(71) Applicant: Hans M. Albertsen, Salt Lake City, UT (US)

(72) Inventor: Hans M. Albertsen, Salt Lake City, UT (US)

(21) Appl. No.: 15/381,319

(22) Filed: Dec. 16, 2016

(51) Int. Cl. A61K 31/436; A61K 31/4745; A61K 31/519; A61K 38/07; A61K 31/5377; A61K 9/20; A61K 31/496

(52) U.S. Cl. CPC A61K 31/436; A61K 9/20; A61K 31/4745; A61K 31/496; A61K 38/07; A61K 31/5377; A61K 31/519

(57) ABSTRACT

The present invention is a method of treating ETD and in particular a method of treating endometriosis and adenomyosis by administering a compound comprised of Rapamycin or a similar mTOR inhibitor and a hormonal contraceptive. The compound may be a natural compound or a synthetic compound. The compound may be in pill form, transdermal patch form, or vaginal ring form.

### CROSS REFERENCE TO RELATED APPLICATIONS

The following U.S. patent applications are expressly incorporated herein in their entirety by this reference: 20160250234 filed Apr. 29, 2016, 20160271119 filed May 28, 2016, and Ser. No. 15/296,175 filed Oct. 18, 2016.

### FIELD OF THE INVENTION

The present invention relates to a therapeutic and method for treating and preventing endometriosis. The therapeutic preferably comprises a compound having a Rapamycin portion and a hormonal contraceptive portion (hereinafter CRC). The CRC may take for instance the form of a pill, a dermal patch, an injectable solution, a pellet for subdermal implantation, or a suppository.

### BACKGROUND OF THE INVENTION

Endometrial tissue is tissue originating in the endometrium (see Appx A). As used herein, Endometrial Tissue Disease (ETD) shall be understood to be disease characterized by the presence of ectopic endometrial tissue (e.g. mislocated endometrial tissue such as endometrial tissue located in other than the endometrium), and shall specifically include endometriosis and adenomyosis.

Endometriosis is a disease in which tissue that normally grows inside the uterus grows outside the uterus (see Appx B). Endometriosis may include autoimmune endometriosis, mild endometriosis, moderate endometriosis, severe endometriosis, endometriomas, superficial (peritoneal) endometriosis, deep (invasive) endometriosis, ovarian endometriosis, endometriosis-related cancers, and “endometriosis-associated conditions”. For the purpose of this invention the term endometriosis is used to describe any of these conditions.

Endometriosis is most generally defined as the presence of autologous endometrial transplants (glands and stroma) at sites outside of the uterus (ectopic endometrial tissues rather than eutopic or within the uterus). The most common sites where eutopic endometriosis is observed clinically are the ovaries, pelvic peritoneum, uterosacral ligaments, pouch of Douglas, and rectovaginal septum although implants have been identified on other peritoneal surfaces of the abdomen, in the thorax, at the umbilicus, and at incision sites of prior surgeries. (Child T J, Tan S L (2001) Endometriosis: aetiology, pathogenesis and treatment, Drugs 61:1735-1750; Giudice et al. (1998) Status of current research on endometriosis, The Journal of reproductive medicine 43:252-262), (Giudice L C (2010) Clinical practice. Endometriosis. The New England journal of medicine 362: 2389-2398.

Endometriosis is a common gynecologic disorder. The prevalence is difficult to know. It has been estimated that it affects approximately 6-10% of all women (range 1-43%—depending on publication and detection method), 40-60% of women with pelvic pain and 30%-50% of infertile women (Di Blasio et al. (2005) Genetics of endometriosis, Minerva ginecologica 57:225-236; Schindler A E (2004) Pathophysiology, diagnosis and treatment of endometriosis, Minerva ginecologica 56:419-435).

Adenomyosis is a medical condition characterized by the presence of ectopic endometrial tissue (glands and stroma) found (especially abnormally deep) in the uterine muscle (see Appx C).

The most widely accepted mechanism for the pathogenesis of endometriosis involves retrograde menstruation whereby endometrial fragments are transported to the pelvic cavity where they attach and grow via autologous transplantation.

The pathogenesis of endometriosis remains to be fully resolved on the cellular and molecular levels, but a range of contributing factors including hormonal, immunological and inflammatory factors have been proposed as triggers for endometriosis. There is evidence that genes associated with endometriosis are central to the regulation of mesothelial barrier integrity via a cellular mechanism known as epithelial-to-mesenchymal transition (EMT) and that loss of mesothelial barrier integrity is a prerequisite for the establishment of endometriosis (Albertsen and Ward, 2016) Genes Linked to Endometriosis by GWAS Are Integral to Cytoskeleton Regulation and Suggests That Mesothelial Barrier Homeostasis Is a Factor in the Pathogenesis of Endometriosis, Reproductive Sciences doi:10.1177/1933719116660847).

Rapamycin (see Appx D) (also known as Sirolimus) is a macrolide produced by the bacterium Streptomyces hygroscopicus. Several different pharmacologic properties have been identified for rapamycin including antifungal properties, immunosuppressant properties and antiproliferative properties.

The mechanistic target of rapamycin (mTOR) is a serine/threonine kinase that in humans is encoded by the MTOR gene (see Appx E). MTOR has been shown to function as is the catalytic subunit of two structurally distinct complexes: mTORC1 and mTORC2.

mTOR Complex 1 (mTORC1) functions as a nutrient/energy/redox sensor and controls protein synthesis. The activity of mTORC1 is stimulated by insulin, growth factors, serum, phosphatidic acid, amino acids, and oxidative stress. mTOR Complex 2 (mTORC2) has been shown to function as an important regulator of the cytoskeleton through its stimulation of F-actin stress fibers, and a group of genes that include PXN, RHOA, RAC1, CDC42, and PRKCA.

Rapamycin is an mTOR inhibitor and has given name to a class of mTOR inhibitors structurally similar to rapamycin known as Rapalogs including: Temsirolimus (CCI-779), Everolimus (RAD001) and Deforolimus (AP-23573 also named Ridaforolimus) (see Appx F). Another class of mTOR inhibiting compounds that are structurally dissimilar to Rapamycin has been developed and function by competing for the ATP catalytic site of mTOR. This class of mTOR inhibitors that has pharmacological properties similar to those of rapamycin and include mTOR/PI3K dual inhibitors (TPdIs) like dactolisib, BGT226, SF1126, PKI-587 and others. This class also includes mTORC1/mTORC2 dual inhibitors (TORCdIs) like sapanisertib, AZD8055, and AZD2014.

The mTOR inhibitors listed here (Rapalogs, TPdIs and TORCdIs) are examples of substantially functional equivalents of rapamycin and in this application they are considered as functional substitutes for rapamycin (hereinafter a rapamycin functional equivalent of RFE).

Rapamycin is used as an immunosuppressant in kidney transplant patients and as cytostatic treatment of tumors in cancer patients. Treatment with rapamycin has been shown to prevent peritoneal fibrosis in a rat model for peritoneal dialysis by inhibiting the activation of Rho GTPases, thereby protecting mesothelial barrier integrity from the EMT repair mechanism which is an initiating event in the formation periotoneal fibrosis (Xiang, et al., 2016). This mechanism is functionally equivalent to the initiating event in the establishment of endometriotic lesions reported by Albertsen and Ward (2016). Rapamycin has also been shown to significantly attenuate the continued development of endometriotic lesions in a mouse model (Ren, et al., 2016).

Female hormonal contraception refers to a birth control method that acts on the endocrine system (see Appx G). There are two main types of hormonal contraceptive (HC) formulations: combined methods which contain both an estrogen and a progestin, and progestogen-only methods which contain only progesterone or one of its synthetic analogues (progestins). Most commonly the HC is administered in form of a pill taken daily, but some HCs may for instance also take a soluble form for injection, a transdermal patch, a vaginal ring, and an intra-uterine device.

HCs commonly regulate the menstrual cycle to a predictable period of 28 days though other periodicities also exists. HCs have been shown to reduce menstrual cramps and the duration and volume of menstrual bleeding and for this reason they are sometimes prescribed to alleviate conditions characterized by excessive bleeding or painful menses including endometriosis. By co-formulating rapamycin with an HC to form a CRC, the synergistic benefit of coordinated administration of rapamycin and an HC in accordance with the progression of the menstrual cycle is achieved resulting in the precisely managed amount of rapamycin for maximal benefit within minimal side-effects. It shall be noted that the ratio of the rapamycin portion to the hormonal contraceptive portion of a given CRC may be adjusted as needed. For instance, the rapamycin portion to hormonal contraceptive portion ratio may be formulated to suit the specific needs of the various stages of the menstrual cycle.

An exemplary use of a CRC is a conventional administration regimen of a conventional HC (e.g. 28 once a day pills wherein 21 of 28 pills are an HC and 7 of 28 pills are a placebo) except that the last ten of the HCs are substituted for a CRC. Such use may be particularly prophylactically beneficial to for instance a young female having no diagnosis of endometriosis but having a family history of endometriosis. A further exemplary use of a CRC is the administration of a CRC via an epidermal patch to provide for a constant dosage of Rapamycin during the menstrual cycle. Such use may be particularly therapeutically beneficial to for instance a woman with surgically diagnosed endometriosis.

### SUMMARY OF THE INVENTION

The present invention is a method of treating ETD, and in particular endometriosis and adenomyosis, by administering a CRC. The function of Rapamycin is to desensitize the mesothelium from undergoing epithelial-to-mesenchymal transition which is an initiating event of ETD, and to provide a cytostatic environment that prevents established endometrial lesions from further growth. The primary function for the oral contraceptive is to provide a stable menstrual periodicy in which Rapamycin can be administered at specific stages in the menstrual cycle. The CRC is composed to specifically affect cellular remodeling of epithelial membranes and glands that facilitate the development, implantation and or growth of endometrial tissues in a place or manner clinically characterized as ETD. More specifically the treatment is intended to prevent or reverse ETD.

Depending on the degree of ETD present in a patient, or the genetic and environmental risk to which a patient is exposed, a graded range of treatment is anticipated. Younger, asymptomatic patients may be treated by simple or low dose CRCs (low amount or ratio of rapamycin) to maintain good epithelial health and thus reduce the risk for developing ETD, whereas more severely affected symptomatic patients may be treated by complex CRCs (high amount or ratio of rapamycin). Moreover, the ratio and amount of rapamycin may be tailored or adjusted based on the phase of the menstrual cycle. As all individuals have a unique genetic make-up and live in unique environments the CRC formulations may benefit from optimization of the formulations and dosages in a manner dependent on genetic and environmental factors specific to a given patient. Benefits of the disclosed CRC treatment method include for instance prevention of ETD, management of ETD symptoms, regression of ETD lesions, reduction in secondary ETD complications, reduction in ETD associated pain, and reduction in ETD associated infertility.

Risk for developing ETD may be assessed on the basis of any of several means including genetic profile, biomarkers, family history, environmental exposures and infertility or any functional equivalent to any of these means.

In an alternate embodiment, a compound is comprised of a Rapamycin portion and an NSAID portion (hereinafter RNC) and is adapted for use in the perimenstrual period in women that do not use an HC.

It shall also be noted that unless indicated otherwise, the phrase “functional equivalent” as used herein with respect to biomarkers shall mean that a second biomarker is substantially equivalent in its diagnostic and/or prognostic value with respect to a given disease as is a first biomarker's diagnostic and/or prognostic value with respect to the given disease.

### DETAILED DESCRIPTION OF THE INVENTION

The invention disclosed herein is a method of preventing or treating an ETD in a patient by administering to the patient an CRC in an amount effective to prevent ETD from developing in a young or asymptomatic woman and to prevent the further development of ETD in a patient and to treat established lesions. Possible CRCs include both natural and synthetic mTOR inhibitors and natural and synthetic hormonal contraceptives.

In additional to reducing or preventing the symptoms of ETD, a benefit of the disclosed invention is that the disclosed treatment of ETD avoids surgical methods of treatment which is a traditional method of treating ETD. Compared to other methods of treating ETD, the disclosed treatment method has a reduced likelihood of interfering with fertility and a reduced likelihood of inducing hirsutism or other types of hormonally caused androgyny.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A compound for use in treating subjects having or being predisposed to ETD and like disorders comprising an mTOR inhibitor component and a hormonal contraceptive component.

2. The compound of claim 1, wherein said mTOR inhibitor component further defines at least one of rapamycin and an RFE.

3. The compound of claim 2, wherein said RFE further defines at least one of a rapalog, a TPdIs and TORCdIs.

4. The compound of claim 1, wherein said mTOR inhibitor component defines at least one of a natural component and a synthetic component.

5. The compound of claim 1, wherein the form of said compound defines at least one of a pill form, a transdermal patch form, and a vaginal ring form.

6. The compound of claim 1, wherein said mTOR inhibitor component further defines at least one of rapamycin, temsirolimus, everolimus, deforolimus, and an RFE.

7. The compound of claim 1, wherein said mTOR inhibitor component further defines at least one of dactolisib, BGT226, SF1126, PKI-587, sapanisertib, AZD8055, and AZD2014.

8. A compound comprising a rapamycin component and a hormonal contraceptive component.

9. The compound of claim 8, wherein the form of said compound defines at least one of a pill form, a transdermal patch form, and a vaginal ring form.

10. A method of treating a patient comprising administering to said patient a compound comprising an mTOR inhibitor component and a hormonal contraceptive component.

11. The method of claim 10, wherein said administering defines at least one of an oral administration, an intravenous administration, a subcutaneous administration, an intra-muscular injection administration, an intra-uterine administration, and a vaginal ring administration.

12. The method of claim 10, wherein the form of said compound defines at least one of a pill form, a transdermal patch form, and a vaginal ring form.

13. The method of claim 10, wherein said subject is ETD asymptomatic.

14. The method of claim 10, wherein said subject is ETD asymptomatic and is known to have at least one ETD associated risk factor.

15. The method of claim 10, wherein said subject has at least one ETD associated biomarker.

16. The method of claim 15, wherein said at least one biomarker defines the minor allele of at least one SNP.

17. The method of claim 10, wherein said mTOR inhibitor component further defines at least one of rapamycin and an RFE.

18. The method of claim 10, wherein said compound defines a plurality of compound pills, and wherein said administration further defines a once-a-day administration of a compound pill for a predetermined duration of days.

19. The method of claim 18, wherein said method further includes at least one step of preceding said once-a-day administration of a compound pill with a once-a-day administration of a placebo pill for a predetermined duration of days and following said once-a-day administration of a compound pill with a once-a-day administration of a placebo pill for a predetermined duration of days.

20. The method of claim 19, wherein said mTOR inhibitor component further defines at least one of rapamycin and an RFE.
